(19) United States
(12) Patent Application Publication
(10) Pub. No.: US 2024/0156414 A1
(43) Pub. Date: May 16, 2024

(54) Method for Predicting Age from Resting-State Scalp EEG Signals Using Deep Convolutional Neural Networks

(71) Applicant: Neuroscience Software Inc. DBA Brainify.AI, Dover, DE (US)

(72) Inventors: Ivan Mishanin, New York City, NY (US); Mariam Khayretdinova, Dover, DE (US); Alexey Shovkun, Dover, DE (US); Andrey Kirysov, Dover, DE (US); Ilya Zakharov, Dover, DE (US)

(21) Appl. No.: 18/493,521

(22) Filed: Oct. 24, 2023

Related U.S. Application Data
(60) Provisional application No. 63/380,719, filed on Oct. 24, 2022.

(51) Int. Cl. A61B 5/00; A61B 5/31; A61B 5/372

(52) U.S. Cl. CPC A61B 5/7267; A61B 5/0006; A61B 5/31; A61B 5/372; A61B 5/7475

(57) ABSTRACT

A method developed for predicting brain age of a human subject as an output of a deep learning model trained on resting state EEG data, which is when the brain is not performing any strenuous mental activity. To improve accuracy, resting state may be measured with both the eyes opened and closed. By automatically extracting relevant EEG result features, a deep learning model is used to calculate the brain age based on the certain markers in the EEG recordings. The primary embodiment of the invention uses a cloud-based service to implement the deep learning algorithm, data augmentation, channel rolling, and model attention highlight algorithms to identify and highlight EEG segments that the model uses for predictive purposes.

### FIELD OF THE INVENTION

The present invention relates generally to a method predicting age from EEG signals. More specifically, the present invention is a method using deep convolutional neural network architecture to process and train on a dataset of resting state electroencephalogram measurements from a plurality of human subjects and thereby predict the brain age of any arbitrary human subject based on the output of the trained model and EEG measurements of the arbitrary human subject.

### BACKGROUND OF THE INVENTION

Brain imaging has long been used in attempts to diagnose neurological, psychiatric or psychological problems. Testing began with MRIs, which provide an understanding of spatial characteristics of the brain's activity, but do not provide the good temporal resolution of high-frequency activity. EEGs have since become the primary method of measuring fast electrical processes in the brain. Compared to the entire brain structure, electrical firings in the cortex provide a much better understanding of the brain's function and capabilities. Medical professionals regularly rely on electroencephalogram, or EEG, measurements to monitor electrical activity in the brain and detect abnormalities. By studying EEG results of healthy individuals, an estimated brain age may be established based on typical brain activity for subjects based on the actual age of control subjects with no major detectable neurogenerative conditions. An individual's EEG results may then be compared against the dataset for the control subjects to determine if the individual may be suffering from a mental or neurodegenerative condition that could cause cognitive decline. Existing implementations use some algorithms to classify EEG results and perform regression tasks, but the existing implementations do not fully consider modern deep learning techniques that may be used to improve data analysis and brain age prediction.

The human aging process occurs on many levels. Particularly, the impact of aging on the brain can be observed throughout the lifespan. The structural connectivity between the hemispheres and functional connectivity (FC) between distinct regions in the brain increase during aging (Madden et al., 2020). FC represents the spatial-temporal correlations between brain networks observed in a task or resting state conditions (Di & Biswal, 2015). At a certain point (around 65 years of age), a gradual decline in FC is observed in normal aging (Madden et al., 2020; Siman-Tov et al., 2017). According to Siman-Tov et al. (2017), brain maturation after the age of 65 has a pronounced impact on connection strength across regions, making some connections weaker, which coincides with the early stages of numerous cognitive dysfunctions.

Some people with mental health conditions are more prone to experience neurological and cognitive dysfunctions early on. For instance, there is growing evidence for FC abnormalities in individuals with depression, bipolar disorder, schizophrenia, as well as neurodegenerative conditions (Albano et al., 2022; Bresnahan et al., 1999; Metzen et al., 2022; Oh et al., 2019). For example, major depressive disorder has been linked to a more prevalent and hyper-connected default mode network (Tang et al., 2022; for a meta-analysis, see Kaiser et al., 2015). Critically, recent studies have highlighted variability between chronological age and accelerated brain aging in people with mental disorders and early life stress (Dunlop et al., 2021; Herzberg et al., 2021). The severity of abnormal fluctuations in FC compared to scans of healthy individuals can be used to identify internalized processes such as abnormal brain aging that does not match chronological age (Dunlop et al., 2021). These alterations in cortical dynamic properties have been linked to cognitive dysfunctions observed across neuropsychiatric conditions (Dunlop et al., 2021). This finding has led to the hypothesis that the age of the brain may serve as a biomarker to diagnose certain mental and neurodegenerative conditions early on.

Previous research has observed age-related brain changes using different methods, such as electroencephalography (EEG), MRI, functional MRI (fMRI), and positron emission tomography (PET) (Bresnahan et al., 1999; Dimitriadis & Salis, 2017; Dunlop et al., 2021; Rajkumar et al., 2021; Zoubi et al., 2018). It is important to note that while MRI-based methods have high spatial resolution imaging, they lack the temporal precision of EEG. EEG is also by far the safest and most widely available method (Rajkumar et al., 2021). For example, unlike PET, it is safe to administer, as it does not include any radiation risks. Additionally, compared with fMRI, EEG is significantly cheaper and easier to use.

EEG methods are widely used to record brain activity during the state of rest (rsEEG), cognitive and motor actions, also known as event-related potentials, and sleep. According to Dimitriadis and Salis (2017), reproducible patterns of accelerated brain age can be observed across various frequency bands in resting conditions, indicating the importance of intrinsic brain oscillations.

Much attention has been drawn to low-frequency alternations in FC observed in rsEEG in people with mental health disorders (Metzen et al., 2022). For example, studies in depression have shown abnormal values of FC dynamics in the prefrontal-limbic regions and abnormalities in the alpha power band at rest (Jaworska et al., 2012; Metzen et al., 2022). Therefore, understanding the EEG FC dynamic and capturing the mechanism behind accelerated brain aging in people with mental conditions could potentially shed light on correct diagnosis, in-time intervention, and early remission onset.

Overall, a limited number of studies have assessed rsEEG recordings to predict brain age (Dimitriadis and Salis, 2017; Zoubi et al. (2018). Both studies relied on quantitative EEG features processed by traditional machine learning algorithms. Zoubi et al. used a general linear model, while Dimitriadis and Salis employed support vector regression to evaluate brain age prediction. However, approaches based on automated feature generation such as deep convolutional neural networks show better results than traditional machine learning.

DCNN has shown promising results in pattern recognition and computer vision applications (Alzubaidi et al., 2021; Sharma et al., 2018; Yamashita et al., 2018). This is due to their ability to automatically extract significant spatiotemporal features that best represent the data from their raw form without preprocessing or human decisions in selecting these features (Olah et al., 2017; Zeiler & Fergus, 2013). Owing to these properties, convolutional networks have solved many medical problems, including diagnosis of brain tumors by MRI (Çinar & Yildirim, 2020; Irmak, 2021) and lung diseases by X-ray images (Bharati et al., 2020; Singh et al., 2021). They have also been used to solve the image segmentation problem (segmenting non-overlapping image areas that have unique features) for medical images, highlighting experts' areas of interest (Feng et al., 2020). Recently, DCNNs have been used to identify biomarkers and diagnose mental disorders by using computer tomography and MRI images (Noor et al., 2020; Vieira et al., 2017). Finally, deep learning has successfully been used to solve tasks related to predicting mental diseases from resting-state EEG recordings (Li et al., 2020; Oh et al., 2019; Sundaresan et al., 2021; Sun et al., 2021) and to predict the sex of the brain (Bučková et al., 2020; Putten et al., 2018). Thus, deep learning is a promising technology for extracting information from a complex data source, such as human brain EEG, without the need for manual feature engineering.

### SUMMARY OF THE INVENTION

A patient's brain age is determined by the output of a deep learning model trained on resting state EEG data, which is when the brain is not performing any strenuous mental activity. To improve accuracy, resting state may be measured with both the eyes opened and closed. By automatically extracting relevant EEG result features, a deep learning model is used to calculate brain age based on certain markers in the EEG recordings. The primary embodiment of the invention uses a cloud-based service to implement the deep learning algorithm, data augmentation, channel rolling, and model attention highlight algorithms to identify and highlight EEG segments that the model uses for predictive purposes. An example of the primary embodiment of the invention in practice is presented in this disclosure. The tests described and parameters decided should be known to represent a single embodiment of the invention used for a study, and the parameters used are a single chosen example not intended to limit scope of other possible parameters that may be used.

### BRIEF DESCRIPTION OF THE DRAWINGS

FIG. 1 is a block diagram illustrating a system overview of the present invention.

FIG. 2 is a flowchart describing an overall process followed by the method of the present invention.

FIG. 3 shows an example of splitting patient data for 10-fold cross-validation.

FIG. 4 shows the deep convolutional neural network model structure. The convolutional layers of the central part of the model have stride (1, 3) and the following kernel sizes: (7, 64), (7, 32), (7, 16), (7, 8). The number of channels changes from 16 to 128, doubling each time.

FIG. 5 shows the correlation between age and EEG band power. False discovery rate (FDR)-corrected significant correlations are marked with a black dot. Color represents the strength of the (non-parametric) Spearman's correlation coefficient.

FIG. 6 shows how dependence of model quality correlates with segment duration (x-axis). The bar chart (left y-axis) shows the number of segments after removing artifacts. The line chart (right y-axis) shows the upper bound of 95% confidence interval for the mean absolute error (MAE) metric of the model.

FIG. 7 is a table showing performance of models predicting brain age trained for different eye states.

FIG. 8 is a table showing the results of the present invention compared to previous works on the topic. The Pearson correlation coefficient for samples of true and predicted values is 0.9.

FIG. 9 is a table of age groups found by the evolutionary algorithm. Note: Square brackets indicate that the end of the range is inclusive, parenthesis—the end is exclusive.

FIG. 10 shows an example of three age groups obtained by the evolutionary algorithm. The true age is marked on the x-axis, and the y-axis shows the difference between the predicted and true age. Orange and blue dots show the prediction errors of the models, trained using the MAE and MALE loss functions, respectively.

FIG. 11 shows model metrics for different age categories. The table shows the found age groups, their size, and the MAE metric (lower is better) of two models trained with different loss functions. Note: Square brackets indicate that the end of the range is inclusive, parenthesis—the end is exclusive.

FIG. 12 shows an example of an attribution map for one EEG segment.

FIG. 13 shows the feature importance score based on integrated gradients attribution for different sexes and eye states aggregated over all EEG segments.

FIG. 14 shows density plots for male and female sexes and different eye states for each EEG channel.

FIG. 15 is a flowchart showing a high-level method for normalizing and evaluating EEG data.

### DETAIL DESCRIPTIONS OF THE INVENTION

All illustrations of the drawings are for the purpose of describing selected versions of the present invention and are not intended to limit the scope of the present invention.

Brain age prediction has been shown to be clinically relevant, with the errors in the prediction associated with various psychiatric and neurological conditions. The present invention is a method primarily developed for creating a new deep learning solution for brain age prediction using raw resting-state scalp EEG.

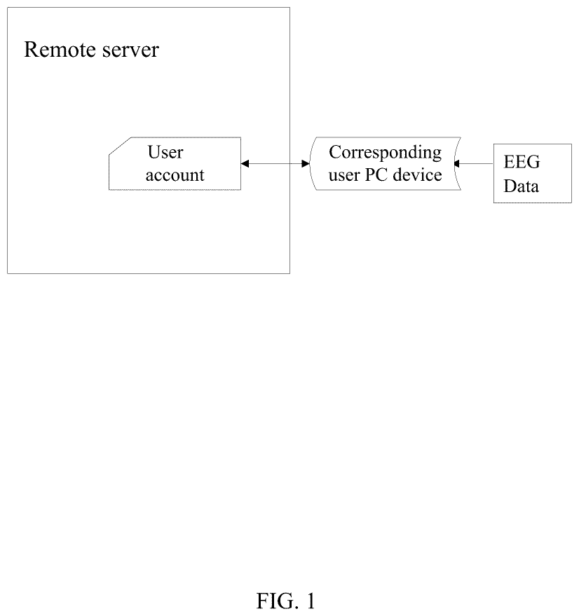

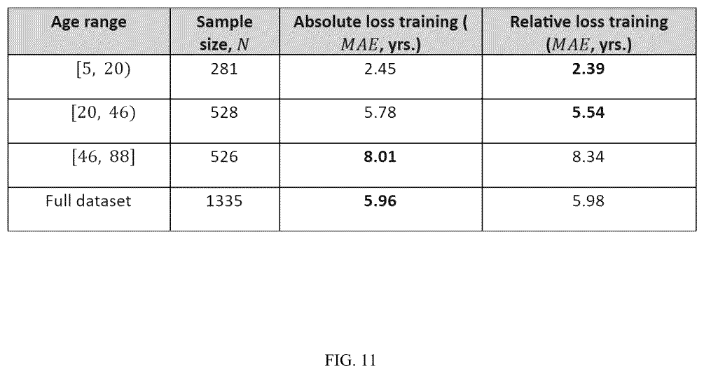

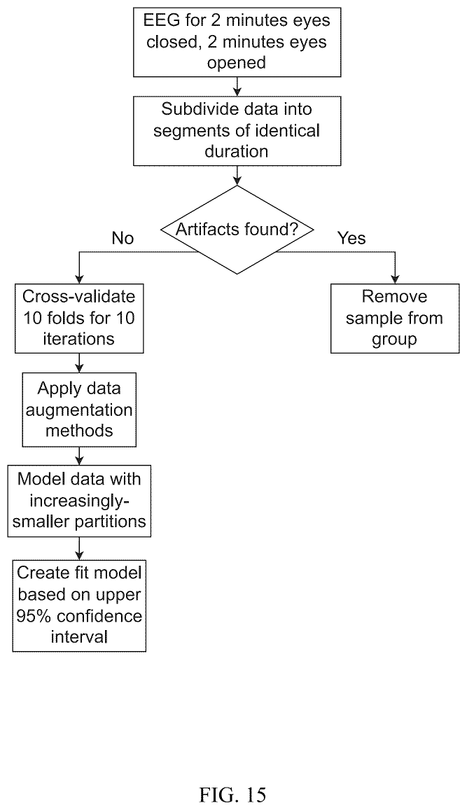

The following description is in reference to FIG. 1 through FIG. 15. As can be seen in FIG. 1, the system used to execute the method of the present invention allows the present invention to function as a tool that enables brain age prediction. To accomplish this, the method of the present invention provides at least one user account managed by at least one remote server, wherein the user account is associated with a corresponding personal computing (PC) device (Step A). The corresponding user PC devices used to interact with the present invention can be, but is not limited to, a smart-phone, a laptop, a desktop, or a tablet PC, or any apparatus with an operating system compatible to access the cloud-based software platform. Further, the corresponding PC device may be connected to a processing unit, an EEG data reception unit, a data transmission unit, and a data storage unit. The remote server is used to facilitate communication between a plurality of such user accounts. Moreover, the remote server is used to execute a number of internal processes for the present invention and is used to store message data. Additionally, the system used to execute the method of the present invention comprises a cloud-based HIPAA complaint software platform capable of storing EEG data of a plurality of human subjects. For example, the cloud-based remote server(s) stores data regarding each corresponding PC device, patient's personal information, health records, insurance information and the desired time slots. The corresponding user PC device allows the user account to perform computer functions such as entering, logging, organizing EEG data, etc.

Brain imaging has long been used in attempts to diagnose problems. Testing began with MRIs, which provide an understanding of spatial characteristics of the brain's activity, but do not provide accurate estimation of high-frequency processes related to electrical firings in the cortex. EEGs have since become the primary method of measuring the fast electrical properties of the brain. Thus, to predict brain age with deep learning algorithms, the electroencephalograph is deployed.

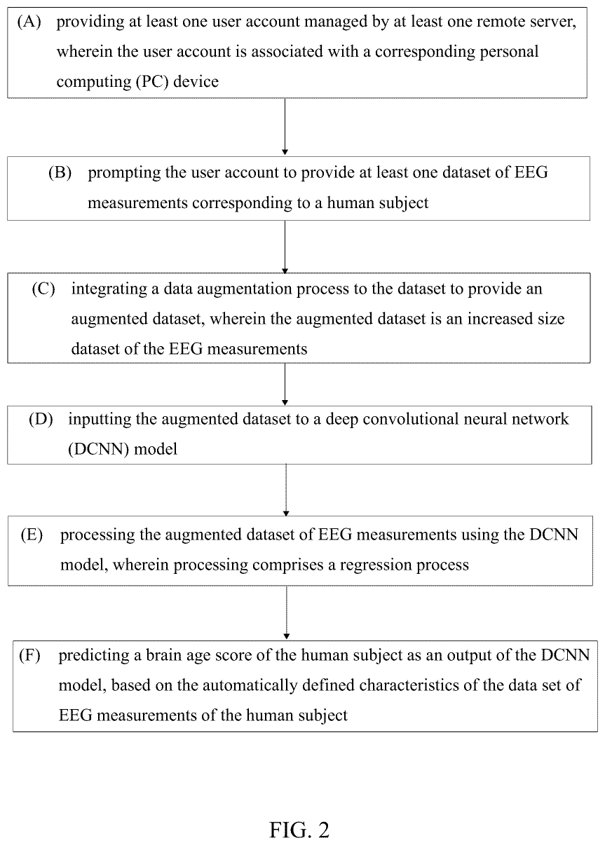

As can be seen in FIG. 2, the overall method of the present invention accomplishes the above-described functionalities by first prompting the user account to provide at least one dataset of EEG measurements corresponding to a human subject (Step B). The electroencephalogram is a non-invasive tool used to measure the brain's activity. The measurement is recorded through the electrodes attached to the individual's scalp. EEG is a relatively fast method to administer temporal resolution with high precision in resting-state brain activity. Moreover, it is cost-effective and allows to perform a measurement in a natural setting, unlike most other tools. The human subject can be any arbitrary person who is willing to undertake the EEG measurements.

The present invention also provides a novel way to automatically extract essential features from raw EEG recordings without manual feature generation that are later used for age of the brain identification. Deep Convolutional Neural Network (DCNN) is used to learn the discriminative time and spatial features from the raw EEG recordings, and Single-Layer Perceptron is applied to solve the regression task. Deep learning models are prone to overfit when trained on insufficiently large datasets. Further, the present invention demonstrates an algorithm for data augmentation that adapts the well-known image processing approaches to raw EEG recordings processing to address this issue. Thus, the overall method of the present invention continues by integrating a data augmentation process to the dataset to provide an augmented dataset, wherein the augmented dataset is an increased size dataset of the EEG measurements (Step C). This is because, despite improvements in understanding the brain from EEGs, deep learning on this information has lagged behind. Existing implementations tend to not use data augmentation, a technique invented in computer vision tasks, to artificially increase dataset size. Augmentations include applications of gaussian noise to an input tensor, random dropout of consequent time-points in EEG channels of the input tensor data, random amplification of the input tensor, shrinking or stretching the time axis with a factor uniform distribution, and applying inverse time flow for all EEG channels. The data augmentation allows to obtain a model with better generalization ability and as a result better accuracy on a smaller training dataset.

The data augmentation method of the preferred embodiment is defined by the following algorithm:
with a probability of 50%, apply gaussian noise to the input tensor with random standard deviation drawn from a uniform distribution (0,1] μV.
with a probability of 70%, apply random dropout of consequent time-points in K EEG channels the input tensor data, where K and Bₖ are drawn from uniform distributions [1, 8] and [1, Len_{seg}*SFreq*0.9], respectively, where Len_{seq} is the length of one EEG segment in seconds and is the sampling frequency of raw EEG data.
with a probability of 50%, apply random amplification of the input tensor with a multiplier drawn from a uniform distribution [0.8, 1.2] for each EEG channel.
With a probability of 50%, shrink or stretch time axis with a factor uniform distribution [0.8, 1.2].
With a probability of 50%, inverse time flow for all EEG channels.

According to the preferred embodiment, the overall method of the present invention continues by inputting the augmented dataset to a deep convolutional neural network (DCNN) model (Step D). The proposed DCNN architecture and training method improve state-of-the-art metrics in the age prediction task using raw resting-state EEG data by 13%. Given that brain age prediction might be a potential biomarker of numerous brain diseases, inexpensive and precise EEG-based estimation of brain age will be in demand for clinical practice.

The overall method of the present invention further continues by processing the augmented dataset of EEG measurements using the DCNN model, wherein processing comprises a regression process (Step E). Further, the present invention also provides a novel way to automatically extract essential features from raw EEG recordings without manual feature generation that are later used for age of the brain identification. The DCNN model is used to learn the discriminative time and spatial features from the raw EEG recordings, and a Single-Layer Perceptron is applied to solve the regression task. In other words, the DCNN model integrates the feature extraction and regression processes into a single automated architecture. Furthermore, the overall method of the present invention continues by predicting a brain age score of the human subject as an output of the DCNN model, based on the automatically defined characteristics of the data set of EEG measurements of the human subject (Step F). In other words, brain age information is successfully extracted from EEG signals with the DCNN model.

A more detailed description of the present invention follows.

According to the preferred embodiment, a subprocess of the method of the present invention comprises taking EEG measurements using resting state eyes closed condition for the human subject, and taking EEG measurements using resting state eyes open conditions for the human subject. It is vital to measure the resting-state brain activity to denote intrinsic neural activity, which is not task-dependent. Resting-state connectivity can be defined as a considerably correlated activity between functionally related brain regions without any stimulus/task. Functional specificity has been shown for the brain resting state activity with closed eyes and open eyes. Further, using of both resting state eyes open and eyes closed conditions for EEG measurements enable to increase accuracy of the brain age score prediction for the human subject. In other words, in the present invention, eyes closed, and eyes open conditions are used in addition to each other that make it possible to achieve higher accuracy in the brain age estimation.

The present invention is aimed at creating a new deep learning solution for brain age prediction using raw resting-state scalp EEG. To this end, the present invention utilized the TD-BRAIN dataset, including 1265 subjects (both healthy controls and individuals with various psychiatric disorders, with a total of 1,335 recording sessions). To achieve the best age prediction, the present invention used data augmentation techniques to increase the diversity of the training set and a developed deep convolutional neural network model. The model's training took place with 10-fold cross-subject cross-validation, with the EEG recordings of the subjects used for training not considered to test the model. In training, using the relative rather than the absolute loss function led to a better mean absolute error of 5.96 years in the cross-validation. Thus, it was found that the best performance can be achieved when both eye open and eyes closed states are used simultaneously. The frontal and central electrodes played the most important role in age prediction. It should be noted that any number of electrodes that are known to one of ordinary skill in the art may be utilized for obtaining the data set of EEG measurements. In other words, a number of electrodes used for EEG measurements ranges between 8 and 24.

DCNNs are known to learn worse due to a well-known “small receptive field” problem. To address it, it is proposed the inventive “channel rolling” method, which extends the receptive field of the first convolutional layer of the network to all input EEG channels. In other words, according to the preferred embodiment, the method comprises a subprocess, wherein a channel rolling process is integrated into the DCNN model before processing the dataset through the DCNN model. Preferably, the channel rolling process extends the receptive field of the first convolutional layer of the network to all input EEG channels.

The channel rolling method that extends the receptive field of the first convolutional layer of the network to all input EEG channels is defined by the following algorithm:

```
xᵢₙ - the input tensor with shape(xᵢₙ) = [1, N_{channels}, Len_{seq} *
SFreq] , where Len_{seq} is the length of one EEG segment in seconds
and SFreq is the sampling frequency of raw EEG data, and N_{channels} >=
8 is the number of scalp EEG channels.
${N}_{steps} = [ \frac{{N}_{channels}}{{KernelSize}_{channels}} ] , the resulting number of channels in the$
output tensor dimension #0, where KernelSize_{channels} is the size
of the kernel of the first convolutional layer for the EEG channels
dimension.
xₒᵤₜ = xᵢₙ, xᵢ = xᵢₙ
For i in [2 .. Nₛₜₑₚₛ]:
  xᵢ = roll(xᵢ, KernelSize, 1) roll tensor xᵢ by KernelSize
  shifts along EEG channels dimension #1.
  xₒᵤₜ = concatenate (xₒᵤₜ, xᵢ, 0) - concatenate tensors xₒᵤₜ and
  xᵢ in the dimension #0.
  return xₒᵤₜ
```

The Deep Convolutional Neural Network of the present invention has the following architecture:

$$Conv ( KernelSize = ( {KS}_{o} , {KS}_{1} ) , kernels = 16 , stride = ( 1 , {Stride}_{time} ) ) \\ BatchNorm ( ) \\ Activation ( ) \\ Conv ( KernelSize = ( {KS}_{o} , [ \frac{{KS}_{1}}{2} ] ) , \\ kernels = 32 , stride = ( 1 , {Stride}_{time} ) ) \\ BatchNorm ( ) \\ Activation ( ) \\ Conv ( KernelSize ) = ( {KS}_{o} , [ \frac{{KS}_{1}}{2} ] ) , \\ kernels = 32 , stride = ( 1 , {Stride}_{time} ) ) \\ BatchNorm ( ) \\ Activation ( ) \\ Conv ( KernelSize = ( {KS}_{o} , [ \frac{{KS}_{1}}{4} ] ) , \\ kernels = 64 , stride = ( 1 , {Stride}_{time} ) ) \\ BatchNorm ( ) \\ Activation ( ) \\ Conv ( KernelSize = ( {KS}_{o} , [ \frac{{KS}_{1}}{8} ] ) , \\ kernels = 128 , stride = ( 1 , {Stride}_{time} ) ) \\ BatchNorm ( ) \\ Activation ( ) \\ Global AvgPool ( ) \\ Linear ( in = 128 , out = 1 )$$

### Where:

the kernel size KS₀ takes one value from the range [2, 10];
KS₁ takes one value from the range [[SFreq/10], SFreq], where SFreq is the sampling frequency of raw EEG data;
stride step along time axis Strideₜᵢₘₑ takes one value from the range [3, 10].

Further, the method comprises a subprocess, wherein a model attribution algorithm is integrated into the DCNN model after processing the augmented dataset. Preferably, the model attribution algorithm enables to identify and highlight EEG segments informative for age estimation. Furthermore, a cloud-based service is provided to implement at least one of the data augmentation processes and the channel rolling processes, through the DCNN model. More specifically, a software as a service (SAAS) framework for accurate brain age estimation prediction suitable for real-time operation is incorporated in one or more embodiments. Accordingly, the client software collects resting state scalp EEG and sends it to the cloud-based Service. The Service processes the request in real-time mode and returns the resulting brain age estimate. The corresponding patient PC device allows the user account to access the platform and generate the required information that will be relayed to the cloud-based remote server(s) and to a corresponding healthcare provider PC device. Preferably, the software platform that runs the method of the present invention is a standalone system and a web app or traditional app, but the software program can be modified to work as an app.

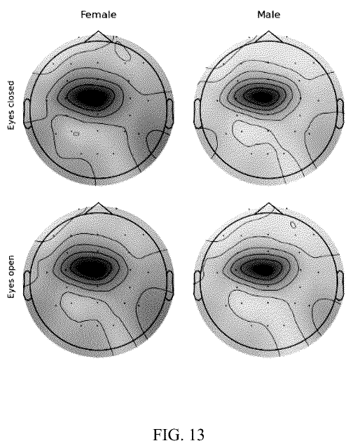

In reference to FIG. 13, the present invention enables generation of activation maps for EEG signals from the DCNN model, wherein activation maps may be used as an alternative to more widespread methods that estimate feature importance for deep learning models. In other words, the present invention showed the feasibility of an activation maps approach to finding the exact features that deep learning models use for brain age prediction.

A more detailed description of the background training process of the model, preprocessing, and other studies and experiments that went into deriving the method of the present invention follows.

For the purpose of understanding the Resting state EEG brain age prediction method and service based on deep learning, references are made in the text and supplementary materials to exemplary embodiments of the Resting state EEG brain age prediction method and service based on deep learning, only some of which are described herein. It should be understood that no limitations on the scope of the invention are intended by describing these exemplary embodiments. One of ordinary skill in the art will readily appreciate that alternate, but functionally equivalent components, materials, designs, and equipment may be used. The inclusion of additional elements may be deemed readily apparent and obvious to one of ordinary skill in the art. Specific elements disclosed herein are not to be interpreted as limiting but rather as a basis for the claims and as a representative basis for teaching one of ordinary skill in the art to employ the present invention.

### EEG Signal Preprocessing

Using an established automatic preprocessing (Brain clinics resources, 2022), noise and other artifacts (e.g., eye blinks or muscle activity) are removed from raw EEG recordings. First, data is bandpass-filtered between 0.5 and 100 Hz, and the notch-frequency of 50 or 60 Hz is removed. Next, the bipolar EOG is calculated and extracted from the EEG signal by using the method proposed by Gratton et al. (1983). In the final stage, the following artifacts are detected using various algorithms: EMG activity, sharp channel-jumps (up and down), kurtosis, extreme voltage swing, residual eye blinks, extreme correlations, and electrode bridging (Al Schuler et al., 2014). If an artifact is found in the EEG recording, then a mark is put on an additional channel, which was used to remove the segment.

In the TD-BRAIN dataset, EEG recordings are 2 minutes in length, in turn indicating a considerable probability of the appearance of artifacts, especially in the EO state. To receive a high-quality sample, all records are divided into segments of identical duration with an overlap and a step equal to 1 second. At the same time, the segment is removed from the sample if there is information about the presence of artifacts on the channel received at the preprocessing phase. Experimentally, the optimal splitting duration of 5 seconds is found, which allows the best quality of model and numerous clean data to be obtained (198,648 segments) (see “Optimal segmentation of EEG recordings” section further).

### Machine Learning Analysis: Cross-Subject Cross-Validation

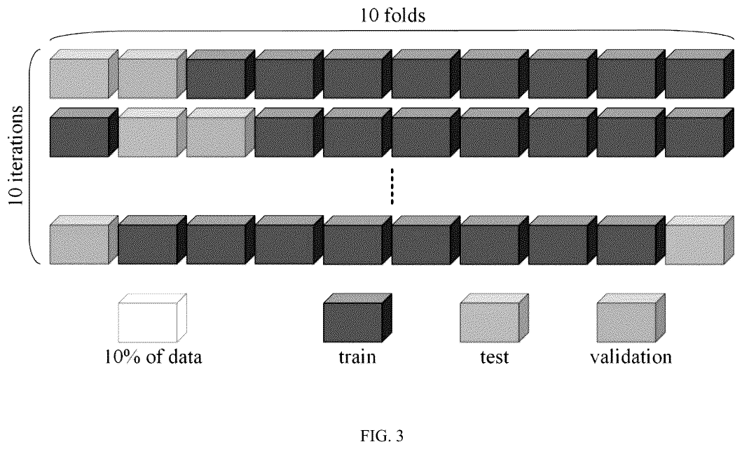

For a correct assessment of the model quality, a 10-fold cross-subject cross-validation with separate validation and testing datasets is used. The cross-validation procedure is repeated multiple times (ten times in the primary embodiment). At each iteration, the whole dataset is divided into ten parts, where eight parts are used for training the network, one for validation during training, and one for testing the final model. An example of splitting is shown in FIG. 3. During training, it is essential to correctly divide the data, as the quality of the model depends on the chosen data split. All EO and EC session segments corresponding to the same subject are placed in the same fold; thus, the model is to detect patterns among different EEG recordings, and not memorize sessions.

Machine Learning Analysis: Data augmentation

To increase the training dataset size and improve the model's quality, the following transformations were applied with experimentally identified parameters to preprocessed EEG recordings as the data augmentation technique:
with a probability of 50%, apply gaussian noise to the input tensor with random standard deviation drawn from a uniform distribution [0, 1] μV;
with a probability of 70%, apply random dropout of Bₖ consequent time-points in k EEG channels of the input tensor data, where k and Bₖ are drawn from uniform distributions [1, 8] and [1, 1800], respectively;
with a probability of 50%, apply random amplification of the input tensor with a multiplier M_{ch} drawn from a uniform distribution [0.8, 1.2] for each EEG channel ch;
with a probability of 50%, shrink or stretch time axis with a factor uniform distribution [0.8, 1.2];
with a probability of 50%, inverse time flow for all EEG channels.

### Machine Learning Analysis: Model

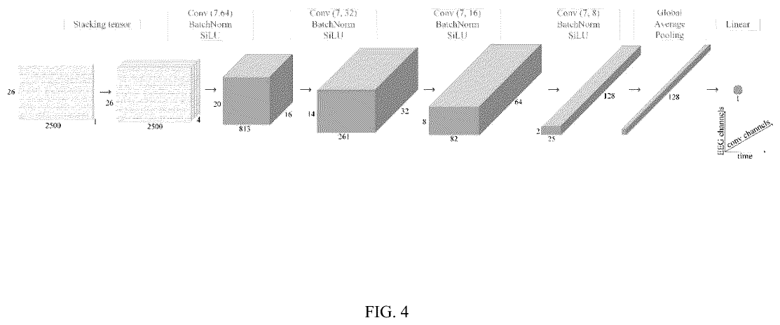

Using a DCNN with a segment of the EEG recording as an input, the segment is transformed into a stacked tensor, shown in FIG. 4, to increase the receptive field of the first convolutional layer. The transformation takes a tensor with dimensions (1, 26, 500*5) as input for a 26-channel 5-second EEG segment. Then, using a cyclic permutation of channels from top to bottom and concatenating them, a new tensor of dimensions (4, 26, 500*5) is made. The central part of the model is four blocks, consisting of a convolutional layer, a batch normalization, and an activation function. The convolutional layer processes the signal with learning weights and resizes the input tensor. The batch normalization technique (Ioffe & Szegedy, 2015) is used to speed up the training of the model and to add regularization by normalizing the data. Sigmoid linear unit is used as an activation function across the convolution layers to add nonlinearity, ensure robustness against noise in the input data, and achieve faster back propagation convergence (Elfwing et al., 2017). After the main blocks, global average pooling is applied to the tensor, transforming the multidimensional tensor into a one-dimensional vector. A linear layer at the end of the model is applied to the vector, whose output is a scalar responsible for the predicted age. Age prediction is performed by applying the model to all artifact-free segments of the EEG session for the eyes-open and eyes-closed tasks, with averaging according to expression (1):

$${Age}_{s} = \frac{{\sum }_{i = 1}^{Ns} Age ( i , s )}{Ns} ,$$

where Age(i,s)≥is a predicted age for session s∈{EO, EC}, i=1 . . . Ns, and Ns is the number of segments in session s.

### Machine Learning Analysis: Model Training

The main loss function in solving the regression task is MAE (2). It is suited for the problem of predicting age and is easily interpreted; MAE was used as one of the metrics. The absolute loss function is not always beneficial (see section “Brain age prediction as a classification problem”). Therefore, the mean absolute logarithmic error (MALE) is applied, the function that is the ratio of the logarithm of a true value to the predicted one (3). The MALE is less sensitive to the scale of the data and allows smaller values prediction in a more efficient manner.

$$MAE ( y , \hat{y} ) = \frac{1}{N} {\sum }_{i = 1}^{N} \vert {y}_{i} - {\hat{y}}_{i} \vert \qquad ( 2 )$$

$$MALE ( y , \hat{y} ) = \frac{1}{N} {\sum }_{i = 1}^{N} \vert ln ( {y}_{i} + 1 ) - ln ( {\hat{y}}_{i} + 1 ) \vert = \frac{1}{N} {\sum }_{i = 1}^{N} \vert ln \frac{{y}_{i} + 1}{{\hat{y}}_{i} + 1} \vert , \\ where N is a sample size , \\ and y and \hat{y} are target and predicted vectors of values , \\ respectively . \qquad ( 3 )$$

During cross-validation, random partitioning of the sample and initialization of the weights of the neural network can lead to different values in metrics. Therefore, the method of the present invention used the upper 95% confidence interval (CI_(95%)) of the sample of test metrics from all iterations (4). Some previous studies do not report the MAE metric but do report the coefficient of determination (R\hat{ }2) metric (5), so the method of the present invention also calculated it for comparison of results. R\hat{ }2 indicates the model fit and is, therefore, an indicator of how well outliers is likely to be predicted by the model through a proportion of the target value variance explained by the model. Thus, using the two metrics together will show not only how the model makes predictions on average but also how well it describes data variance.

$${CI}_{95 \%} = \underline{x} + \frac{1.96 \cdot std}{\sqrt{{N}_{cv}}} \qquad ( 4 )$$

$${R}^{2} ( y , \hat{y} ) = 1 - \frac{{\sum }_{i} {( {y}_{i} - {\hat{y}}_{i} )}^{2}}{{\sum }_{i} {( {y}_{i} - \overline{y} )}^{2}} , \qquad ( 5 )$$

$$where \underline{x} is a mean metric value , std is a metric standard \\ deviation , \underline{y} = \frac{{\sum }_{i} {y}_{i}}{N} , 1.96 is the approximate value of the \\ 97.5 percentile point of the standard normal distribution , \\ {N}_{cv} is the number of the cross - validation folds , and the \\ rest of the notation as in formulas ( 2 ) and ( 3 ) .$$

The model was trained with pytorch and catalyst (Kolesnikov, 2018) libraries by using the Adam optimization algorithm (Kingma & Ba, 2017) with a starting learning rate of 3.10\hat{ }(4) and a batch size of 512 segments. Also, the method of the present invention used the ‘reduce on plateau’ scheduler with the patience of three epochs to 5 obtain the maximum quality of the network and the ‘early stopping’ technique after ten epochs without validation metric improvement to prevent model overfitting. The training was performed on four Nvidia A 10G GPUs and took 5 hours on average.

Processing Results—Age Correlations with EEG Band Power

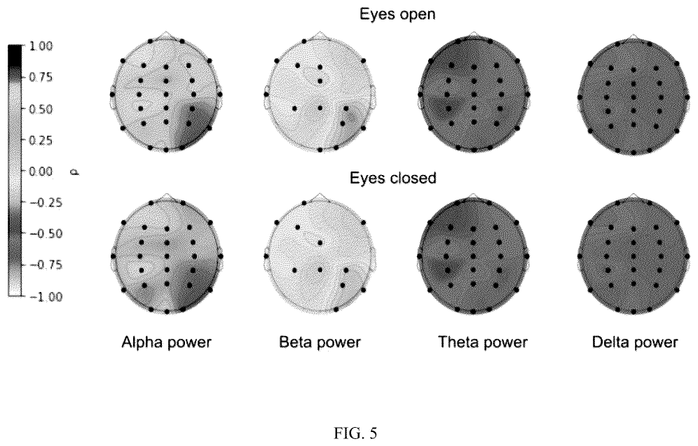

To ensure that EEG signals contain information that can be correctly extracted by the deep learning algorithms prior to brain age prediction, the zero-order correlations between age and EEG band power (alpha: 8-12 Hz, beta: 12-Hz, delta: 1-4 Hz, theta: 4-7 Hz) are calculated separately for each EEG electrode. The power of the bands is also calculated separately for eyes-closed and eyes-open conditions. The results are presented in FIG. 5.

From FIG. 3, it can be seen that EEG power is associated with age for all narrow bands for nearly all electrodes. The highest correlations are found for the absolute delta band power, and the lowest correlations for the absolute beta band power, with the overall decline in EEG power with age across all bands The presence of significant correlations is necessary in building the deep learning model.

### Optimal Segmentation of EEG Recordings

The abundant presence of artifacts in resting-state EEG recordings can deteriorate the quality of the resulting neural network. A frequently used approach is to divide 2-minute recordings for eyes-open and eyes-closed states into segments of several seconds, with subsequent removal of segments with artifacts from consideration.

With this approach, the task of choosing the optimal duration of one segment arises. As the duration of a segment increases, it becomes easier for the neural network to regress to the target variable, as it processes each segment independently of the others. At the same time, deleting a longer segment due to an artifact deprives the neural network of more information compared to a shorter segment. The latter complexity can be partially leveled out by using segments intersecting with a step of one second. The task is formulated as an optimization problem, shown below:

SegLenₒₚₜᵢₘₐₗ=(CrossValMAE(Φ)(X), N_{cv}=10))

X(SegLen)=DataSplit(SegLen, overlap=1),
where CrossValMAE (Φ, N_{cv}) is the cross-validation MAE score calculation for neural network Φ with N_{cv} fold iterations; Φ is the neural network functional; and DataSplit (SegLen, overlap) is an algorithm splitting records into segments of length SegLen with overlap seconds.

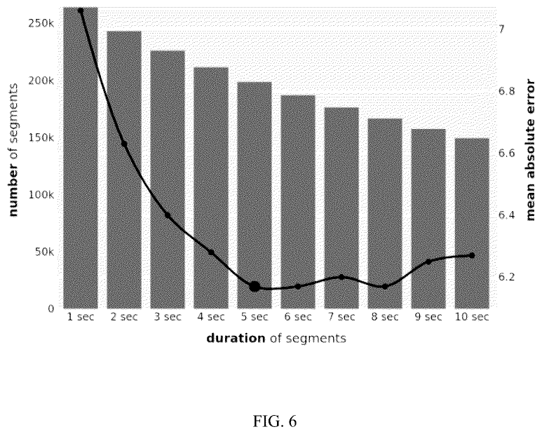

To solve this problem, ten independent models are trained on segments of duration from 1 to 10 seconds (in the case of 1 second, there was no overlap between segments) and evaluated their quality and the sample size after artifact removal, shown in FIG. 6. For reliability, the optimal segment length is chosen based on the upper bound of the 95% MAE confidence interval, which was calculated by cross-validation. The calculated optimal duration of 5 seconds was used for further experiments. This allowed the removal of all segments with artifacts while keeping the total number and duration of segments in training at sufficient levels.

Thus, the final prediction of the brain age of a subject is carried out by predicting the age for all 5-second artifact-free segments from both EC and EO sessions with subsequent averaging of the obtained values.

### Influence of Eye State

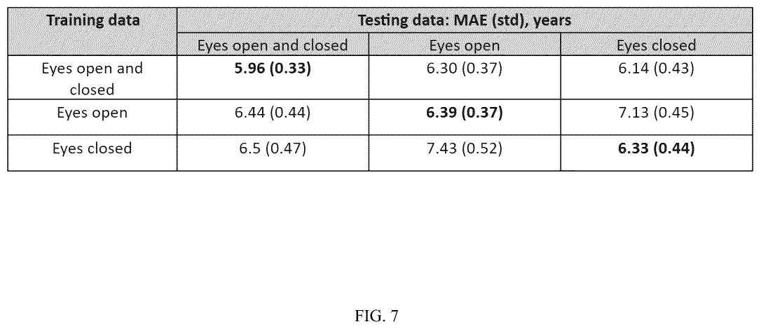

A series of experiments was carried out to study the influence of eye state, during EEG recording, on age prediction. Three DCNNs were trained independently on different datasets: only on data with open eyes, only with closed eyes, and with both conditions. Each of the models predicted these datasets separately, shown in FIG. 7.

As a result, the method of the present invention observed almost identical single-eye-state model performance on the known modality data (MAE was 6.39 and 6.33 for open and closed eyes, respectively). At the same time, the eyes-closed model experienced more difficulty with the opposite eye-state data relative to the eyes-open model (MAE 7.43 versus 7.13). Thus, the open eyes condition was slightly more informative for the DCNN predicting brain age than closed eyes. At the same time, the best performance was achieved using both eye states simultaneously. Both modalities acted as additional data augmentations and provided the DCNN with better performance and generalization ability.

### Accuracy of Brain Age Prediction

The experiment results confirm the presence of brain age information in the resting-state EEG recordings, effectively extracted by a deep convolutional neural network. The proposed DCNN architecture predicts human brain age, with the best-known quality achieved on the resting-state EEG recordings with MAE=5.96 (std=0.33) years and R\hat{ }2=0.81 (std =0.03). All experiments were conducted using robust 10-fold cross-subject cross-validation on a subset of the TD-BRAIN dataset containing resting-state EEG with open and closed eyes.

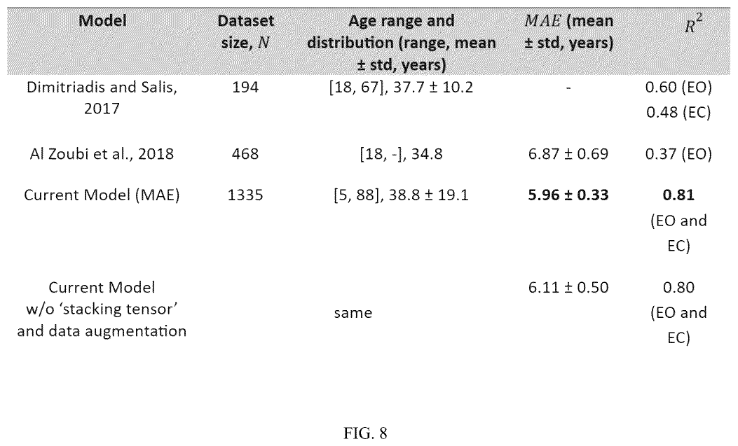

FIG. 8 shows metrics used typically by models predicting brain age. The table shows the size of datasets, mean age and standard deviation (std), as well as the MAE (lower is better) and R2 (higher is better) metrics obtained on the corresponding datasets.

The ‘roll and shift’ method and data augmentation plays a noticeable role in DCNN quality. The first technique allows the first layer of the network to obtain more information from the signal, and the second improves the model's ability to generalize. An increase in the size of the input tensor, and the application of various transformations to the segments of the EEG signal, leads to a MAE metric improvement of 2.5% (from 6.11 (std=0.5) to 5.96 (std=0.33), Table 2). Applying these methods together seems especially useful, as the network should not only be more precise but also possess better generalization ability.

Although age is a continuous variable, some brain studies consider it as a categorical by dividing participants into age groups (Bonnet & Arand, 2007; Bresnahan et al., 1999; Gaudreau et al., 2001). At the same time, different studies use different boundaries between groups. The current model makes it possible to find the optimal partition of the entire age range into K non-overlapping groups. Let y=(y₁, . . . , y_{N}) and ŷ=(ŷ₁, . . . , ŷ_{N})be the target and predicted age, and b₁, . . . bₖ₊₁ are borders for the age groups C₁, . . . , Cₖ such that Cᵢ=[bᵢ, bᵢ₊₁) for i=1 . . . k. The method of the present invention will look for boundaries that increase the balanced accuracy score bAcc(B(y), (B(ŷ)) described in Brodersen et al. (2010), where B(x) is the age matching formula, such as B(x)=Cⱼ if x∈[bⱼ, bⱼ₊₁). The method of the present invention also set restrictions on the class sizes |C| so that the size of the largest class does not exceed the smallest one by a factor of 2, so the classes will be more balanced. Thus, the optimization problem of finding the boundaries of age groups has the following form:

bAcc(B(y), B(ŷ))→b₁>b₂ . . . >bₖ₊₁|Cᵢ|≥|Cᵢ|

The stochastic global search optimization Differential Evolution algorithm (Das & Suganthan, 2011) was used to solve the above inequality. FIG. 7 shows the optimal class boundaries found using the mentioned algorithm for K={2,3,4,5}.

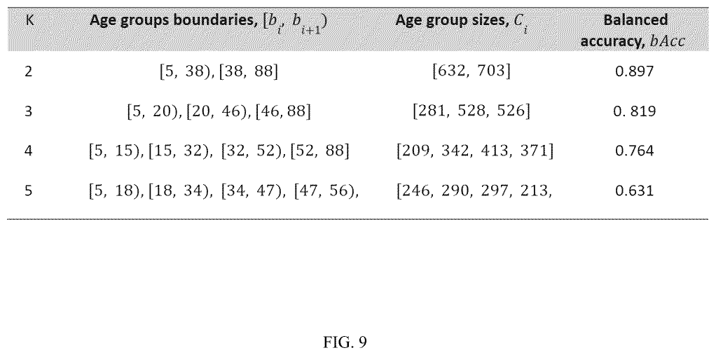

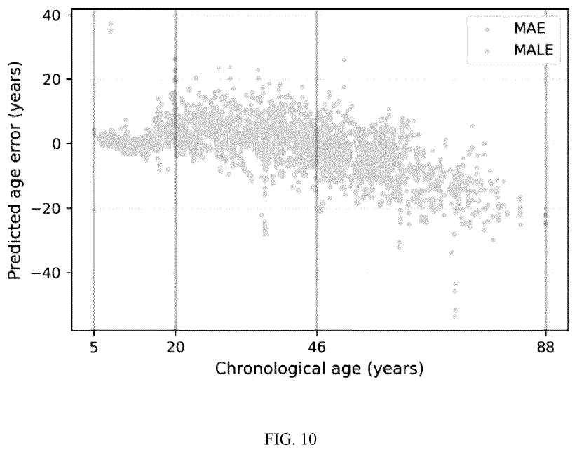

From FIG. 9 and FIG. 10, a very prominent young group aged 5-20 is visible: the model predicts it much more accurately than the middle age. There is also a group with an age of more than approx. 50 years, in which the model consistently errs towards a younger age. This seems logical since the brain develops rapidly at a young age, and a couple of years make a sizable difference, while in old age, a difference of 5-7 years may not be noticeable. These observations lead us to conclude that, from a physiological point of view, it would be most natural to optimize not the absolute error of MAE but rather the relative one, for example, MALE.

Models were trained with both absolute and relative loss functions and compared their mean absolute error metric on the obtained age groups for K=3. Table 4 shows that DCNN trained with relative loss is more valuable for further application. Metrics indicate that using MALE loss function reduces the spread of values in the first two age groups, which makes it possible to predict age more effectively.

### Importance of Cross-Subject Validation

The critical role of the cross-validation strategy used is notable, because it allows an objective assessment of the quality of the model. First, the selected number of folds allows a sufficiently large test set size of more than a hundred sessions. Furthermore, it allows more accurate estimation of the boundaries of the confidence interval in the resulting metric, when compared to a smaller number of folds. Second, cross-subject separation eliminates data leakage. It guarantees the distribution of all information from one session, including open and closed eyes, only inside the training, validation, or testing set. This deprives the neural network of the ability to memorize and use “session fingerprints” for age prediction. The model must extract patterns from the data familiar to different sessions and subjects, ultimately leading to better generalizing ability. To illustrate the possible data leakage effect, the method of the present invention replaced the cross-subject split rule with a random split. The model trained on 10-fold cross-validation with random mixing of session information between folds achieves MAE=2.03 years and R\hat{ }2=0.97. Such metrics look optimistic, but, unfortunately, would not be replicated with new or hold-out EEG sessions.

### Model Explanation

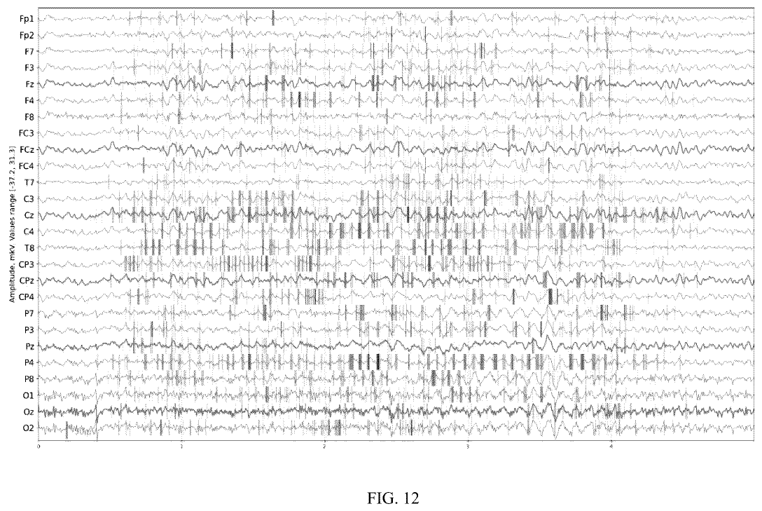

While DCNNs have had a large impact on various tasks, explaining their predictions is still challenging. One approach is to assign an attribution value, which is also called “relevance” or “contribution,” to each input feature of a network. Given a specific target neuron c, the goal of the attribution method is to determine the contribution R_{c}=[_{c}¹ . . . R_{c}^{N}]∈R^{N} of each input feature xᵢ to the output S_{c}. The problem of finding attributions for deep networks has been tackled in several previous works (Bach et al., 2015; Montavon et al., 2017; Simonyan et al., 2013; Springenberg et al., 2014; Zeiler & Fergus, 2013; Zintgraf et al., 2017). In the examined regression task, there is a single output neuron S_{c} responsible for the age prediction. When the attributions of all input features are arranged to have the exact shape of the input sample, attribution or sensitivity maps are created, shown in FIG. 12.

Using the Integrated Gradients method proposed by (Sundararajan et al., 2017) in conjunction with the Smooth Grad method (Smilkov et al., 2017), the sensitivity map is sharpened. Attribution maps are obtained at a segment level and aggregated along the time dimension, providing a feature importance score with [channel, sex, eye-state] resolution for each segment. The average feature importance illustration on a topological head map shows its concentration around the Cz channel and a bit to C1 on the left with a slight difference between the eye states and sex of a subject, as shown in FIG. 13.

This result does not confirm the high feature importance of the left parieto-temporal area (TP9 electrode according to 10-10 System) reported by Zoubi et al. (2018). The difference in most essential regions may be attributed to the difference in approaches: the current study used a DCNN as an automated feature extractor, while the study conducted by Zoubi et al. used a stack-ensemble of classical machine learning algorithms over hand-crafted features. Different machine learning methods can solve the same problem in different ways. The other possible reason is the method used to analyze the operation of the neural network; other algorithms may show different results.

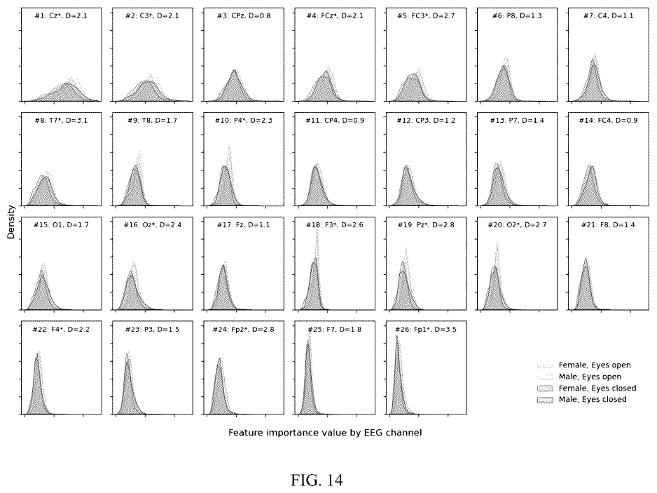

More detailed results can be obtained from FIG. 14, where almost no difference in the feature importance between sexes can be observed, though, with some difference in eye states. Open eyes are shifted to the right, providing slightly more valuable information for the DCNN compared to eyes closed in some but not all channels: Cz, C3, FCz, FC3, etc. Since the network is sensitive to the eyes closed and eyes open states, the method can speculate whether it has learned to use residual EOG information, which could remain after preprocessing. Indeed, the method observed a significant inter-eye-state difference (D≥2.8) in Fp1 and FP2 channels. Typically, they experience the most remarkable influence from EOG. On the contrary, the method of the present invention thinks this is not the case. Further,the method of the present invention believe this is due to these channels being among the least important for the model.

FIG. 14 shows density plots for male and female sexes and different eye states for each EEG channel. Channels are presented in descending order of total attribution with larger (more interesting) values to the right on the x-axis. Channel is marked with ‘*’ when the absolute difference ‘D’ between medians for eyes open and closed attribution is greater than 2*IQR for the eyes-closed condition.

Accurate Brain Age Prediction from EEG Feasibility

The present invention aimed to develop a deep learning model for brain age prediction from resting-state EEG recordings. According to our results, brain age information can be successfully extracted from EEG signals with a DCNN. The method of the present invention used data from eyes open and eyes closed conditions for prediction. The present invention found that while the open eyes condition is slightly more informative for the DCNN to predict brain age than closed eyes, the best performance can be achieved when both eye states are used simultaneously, divided into 5 s epochs. The method of the present invention has also demonstrated a crucial role for correct cross-validation: when applied inappropriately, it can lead to serious inflation of the prediction accuracy. One important result of the study is the introduction of a relative loss function, which works better than the absolute one. Finally, our results also indicate that prediction accuracy can differ for different age groups, with the highest accuracy for the participants 15-20 years old.

The present invention improves the best-known MAE for brain age prediction based on resting-state EEG by 13% (from 6.82 to 5.97 years), and R² by 35% from (0.60 to 0.81). Why was R² increased more than MAE? Presumably, Zoubi et al. (2018) had many outliers and/or their model predicts them poorly. Dimitriadis and Salis (2017), unfortunately, did not report MAE. One important difference between our research and previous work is related to the bigger sample size utilized for the current analysis. It has been recently shown that bigger samples in neuroscience studies are needed for obtaining more stable and reproducible findings (Marek et al., 2022) The improvement in results can be also related to wider age range (the presence of young people under the age of 18) in our dataset. However, MRI-based brain age prediction of MAE is significantly higher. In a recent study, Leonardsen and colleagues (2022) achieved MAE=2.47. However, in their study, the deep learning CNN model was trained on a much bigger sample (N=53,542), leaving the possibility that EEG-based prediction can also be increased with a larger sample. One advantage of EEG brain age prediction compared to MRI brain age prediction is that EEG signals contain high-frequency brain activity, which is crucial for communication within the brain (Fries, 2015). Whether the modality (MRI or EEG) or the sample size is the more important factor in age prediction accuracy is a matter of future studies.

In this study, the present invention has also shown that building from DCNN model activation maps for EEG signals is feasible. The activation maps can be used as an alternative to more widespread methods that estimate feature importance for deep learning models. Detailed analysis of the neurophysiological characteristics of age-related EEG sections, highlighted by the activation maps method, and comparison of it to the results of other methods is a matter for future research.

### Brain Age Prediction as a Potential Biomarker

A promising application of brain age prediction is related to investigating prediction errors. The delta between prediction from brain characteristics and one's chronological age (brain-predicted age difference, brain-PAD) has been previously associated with multiple illness. More extreme brain-PAD was observed in patients with depression (Schmaal et al., 2020), cognitive impairment (Elliott et al., 2021), dementia (Wang et al., 2019), Alzheimer's disease (Gaser et al., 2013), and schizophrenia (Rokicki et al., 2020). In a recent large-scale MRI study, higher brain-PAD was linked to age-related changes in glucose level, insulin-like growth factor-1, level of glycated hemoglobin, and negative lifestyle habits such as smoking or excessive alcohol consumption (Leonardsen et al., 2022). However, the effect size of the association between MRI-based brain-PAD and various health-related problems was relatively small, suggesting cautious causal interpretation. One advantage of EEG brain age prediction compared to MRI brain age prediction is that the EEG signal contains high-frequency brain activity. This fact can play a crucial role when it comes to correlating brain-PAD with neurological and psychiatric disorders because of their functional rather than anatomical nature (Finn & Constable, 2016). Critically, high-frequency brain oscillations contain information about the dynamic synchronization between different brain areas, forming functional brain networks (Fries, 2015). Alterations within brain networks are now seen as the major source of different disorders (Bassett & Bullmore, 2009; Heuvel & Fornito, 2014). One way to further increase both the sensitivity and specificity of EEG brain age prediction and brain-PAD as a functional biomarker can be to account for the network information available in EEG synchronization patterns.

### Alternate Embodiments and Implementations

High-accuracy prediction is feasible with resting-state EEG, which is an important improvement due to the much higher availability and lower cost of EEG technologies. Given that brain-PAD is seen as an important potential biomarker of numerous neurological and psychiatric conditions, its inexpensive and precise EEG-based estimation will be in demand for clinical practice in such areas as automatic diagnostics and treatment predictions. For example, such projects are now developed for depression studies (Zhang et al., 2020). It would be reasonable to conduct further research in several directions. First, to identify factors that allow DCNNs to determine the age of the human brain, to study these factors, and to verify them from a neurophysiological point of view. Second, to create a neural network with a high generalizability, which will make it possible to predict the age of the human brain by using data collected in new conditions (a different site, different equipment, etc.). Third, to explore whether there would be benefits using EEG-informed fMRI (i.e., combining EEG with higher spatial resolution fMRI data). Finally, the model is trained to predict age, but it can also be valuable for transferring identified features from one domain (age prediction in the current study) to another domain (neuropsychiatric disorders). This would allow finding new brain-state biomarkers and predicting treatment outcomes for mental disorders.

The current deep learning model was built on the EEG data of patients with different disorders and has to be tested on normative EEG. However, to our knowledge, large-scale, normative resting-state EEG of a wide age range is at present either absent or unavailable for the research community. Moreover, existing datasets are mostly limited to participants of European ancestry. Creating a large-scale open data set with a diverse sample is a necessary step for further development of EEG brain age prediction models. Another limitation relates to the interpretability of the obtained deep learning model. The present invention showed the feasibility of an activation maps approach to finding the exact features that deep learning models use for brain age prediction. However, alternate embodiments of the invention address the neurophysiological properties of activation maps.

### Conclusion

The DCNN with the introduced loss function outperforms previously used methods by 13% if suitable data augmentation techniques are applied, using proper cross-validation procedures for avoiding inflated prediction accuracy. The development of rsEEG-based brain age prediction is important for clinical applications and can scale up its acceptance in practice.

All illustrations of the drawings are for the purpose of describing selected versions of the present invention and are not intended to limit the scope of the present invention.

Although the invention has been explained in relation to its preferred embodiment, it is to be understood that many other possible modifications and variations can be made without departing from the spirit and scope of the invention.

What is claimed is:

1. A patient-specific brain age score prediction method comprising:
(A) providing at least one user account managed by at least one remote server, wherein the user account is associated with a corresponding personal computing (PC) device;
(B) prompting the user account to provide at least one dataset of EEG measurements corresponding to a human subject;
(C) integrating a data augmentation process to the dataset to provide an augmented dataset, wherein the augmented dataset is an increased size dataset of the EEG measurements;
(D) inputting the augmented dataset to a deep convolutional neural network (DCNN) model;
(E) processing the augmented dataset of EEG measurements using the DCNN model, wherein processing comprises a regression process;
(F) predicting a brain age score of the human subject as an output of the DCNN model, based on the automatically defined characteristics of the data set of EEG measurements of the human subject.

2. The method of claim 1, comprising:
taking EEG measurements using resting state eyes closed condition for the human subject; and
taking EEG measurements using resting state eyes open conditions for the human subject.

3. The method of claim 2, wherein using of both resting state eyes open and eyes closed conditions for EEG measurements enable to increase accuracy of the brain age score prediction for the human subject.

4. The method of claim 1, wherein the DCNN model integrates the feature extraction and regression processes into a single automated architecture.

5. The method of claim 1, wherein a number of electrodes used for EEG measurements ranges between 8 and 24.

6. The method of claim 1, comprising:
integrating a channel rolling process to the DCNN model before processing the dataset through the DCNN model; and
integrating a model attribution algorithm to the DCNN model after processing the dataset.

7. The method of claim 6, wherein the channel rolling process extends the receptive field of the first convolutional layer of the network to all input EEG channels.

8. The method of claim 6, wherein the model attribution algorithm enables to identify and highlight EEG segments informative for age estimation.

9. The method of claim 6, wherein a cloud-based service is provided to implement at least one of the data augmentation processes and the channel rolling process, through the DCNN model.

10. The method of claim 1, comprising:
enabling generation of activation maps for EEG signals from DCNN model, wherein activation maps may be used as an alternative to more widespread methods that estimate feature importance for deep learning models.

11. A patient-specific brain age score prediction method comprising:
(A) providing at least one user account managed by at least one remote server, wherein the user account is associated with a corresponding personal computing (PC) device;
(B) prompting the user account to provide at least one dataset of EEG measurements corresponding to a human subject;
(C) integrating a data augmentation process to the dataset to provide an augmented dataset, wherein the augmented dataset is an increased size dataset of the EEG measurements;
(D) integrating a channel rolling process to the DCNN model before processing the dataset through the DCNN model;
(E) providing a cloud-based service to implement at least one of the data augmentation process and the channel rolling process, through the DCNN model;
(F) inputting the augmented dataset to a deep convolutional neural network (DCNN) model;
(G) processing the augmented dataset of EEG measurements using the DCNN model, wherein processing comprises a regression process; and
(H) predicting a brain age score of the human subject as an output of the DCNN model, based on the automatically defined characteristics of the data set of EEG measurements of the human subject.

12. The method of claim 11, comprising:
taking EEG measurements using resting state eyes closed condition for the human subject; and
taking EEG measurements using resting state eyes open conditions for the human subject.

13. The method of claim 12, wherein using of both resting state eyes open and eyes closed conditions for EEG measurements enable to increase accuracy of the brain age score prediction for the human subject.

14. The method of claim 11, wherein the DCNN model integrates the feature extraction and regression processes into a single automated architecture.

15. The method of claim 11, wherein a number of electrodes used for EEG measurements ranges between 8 and 24.

16. The method of claim 11, comprising:
integrating a model attribution algorithm to the DCNN model after processing the dataset, wherein the model attribution algorithm enables to identify and highlight EEG segments informative for age estimation.

17. The method of claim 11, wherein the channel rolling process extends the receptive field of the first convolutional layer of the network to all input EEG channels.

18. The method of claim 11, comprising:
enabling generation of activation maps for EEG signals from DCNN model, wherein activation maps may be used as an alternative to more widespread methods that estimate feature importance for deep learning models.
